(19) United States
(12) Patent Application Publication
(10) Pub. No.: US 2022/0283186 A1
(43) Pub. Date: Sep. 8, 2022

(54) METHOD FOR DETERMINING ALZHEIMER-TYPE DEMENTIA OR MILD COGNITIVE IMPAIRMENT

(71) Applicant: NIPRO CORPORATION, Osaka (JP)

(72) Inventors: Hiroshi YOSHIDA, Kyoto (JP); Yoshinori KOSOKU, Osaka (JP); Yuka SANO, Shiga (JP)

(73) Assignee: NIPRO CORPORATION, Osaka (JP)

(21) Appl. No.: 17/638,044

(22) PCT Filed: Aug. 28, 2020
(86) PCT No.: PCT/JP2020/032503

§ 371 Date: Feb. 24, 2022

(30) Foreign Application Priority Data
Aug. 30, 2019 (JP) 2019-158882

(51) Int. Cl. G01N 33/68

(52) U.S. Cl. CPC G01N 33/6896; G01N 2800/2814

(57) ABSTRACT

The present disclosure provides a method for detecting Alzheimer's cognitive impairment or mild cognitive impairment, comprising: measuring an amount of homocysteic acid in a biological sample from a subject who suffers or possibly suffers from a neurological disease, and detecting Alzheimer's cognitive impairment or mild cognitive impairment based on the measured amount of the homocysteic acid.

### TECHNICAL FIELD

The present invention relates to a method for determining Alzheimer-type dementia or mild cognitive impairment. More specifically, the present invention relates to a method for determining Alzheimer-type dementia or mild cognitive impairment based on an amount of homocysteic acid in a biological sample. The present invention also relates to a method for determining the progress of Alzheimer-type dementia or mild cognitive impairment. The present invention further relates to a kit for use in the methods.

### BACKGROUND

Mild cognitive impairment (MCI) indicates a precursor state of dementia, most of which Alzheimer's disease (AD) occupies. Not all subjects with MCI will progress to dementia (convert), or some may return to normal (revert). It is said that the revert rate is 14% to 44% (non-patent literature 1). Early detection of MCI and early therapeutic intervention is expected to reduce the risk of dementia.

Various diagnostic criteria are exemplified as Diagnostic criteria for MCI, including the dementia diagnostic criteria in the International Statistical Classification of Diseases and Related Health Problems 10th Edition (ICD-10) provided by the World Health Organization and the dementia diagnostic criteria in the Diagnostic and Statistical Manual of Mental Disorders 5th Edition (DSM-5) provided by American Psychiatric Association (2013).

### CITATION LIST

Non-Patent Document 1: Jennifer J. Manly, et al., Ann Neurol. 2008; 63: 494-506

### SUMMARY

### Technical Problem

The above diagnostic criteria mainly include the diagnosis of cognitive function and the medical interview about life conditions as to whether the cognitive function causes trouble in their social and daily life. In particular, identifying a subject with AD or MCI from subjects suffering or possibly suffering from a neurological disease requires various diagnostic criteria and cannot be easily carried out. Specifically, the AD identification according to Clinical Practice Guideline for Dementia 2017 includes excluding consciousness disorder and depression from suspected dementia in a broad sense (neurological disease), diagnosing and excluding medical diseases and surgical diseases, and further identifying Alzheimer-type dementia according to medical opinions based on clinical conditions, imaging, and examinations.

There is a demand in the technical field to search for a biomarker associated with cognitive decline and develop a method capable of objectively and easily determining mild cognitive impairment.

The present inventors searched for a biomarker with less physical burden capable of determinating Alzheimer-type dementia or mild cognitive impairment in subjects who suffer or possibly suffer from neurological disease and found that the amount of homocysteic acid in blood sample may be used to determine Alzheimer-type dementia or mild cognitive impairment.

### Solution to Problem

In particular, the present invention provides a method for determining Alzheimer-type dementia or mild cognitive impairment in a subject who suffers from or possibly suffers from a neurological disease, a method for determining the developing progress thereof, and a kit for use in the determining method, as described below.

[Item 1] A method for determining Alzheimer-type dementia or mild cognitive impairment, comprising measuring an amount of homocysteic acid in a biological sample from a subject who suffers from or possibly suffers from a neurological disease, and determining Alzheimer-type dementia or mild cognitive impairment based on the measured amount of the homocysteic acid.

[Item 2] A method for determining the progress of developing Alzheimer-type dementia or mild cognitive impairment, comprising: measuring an amount of homocysteic acid in a first biological sample from a subject who suffers from or possibly suffers from a neurological disease; measuring an amount of homocysteic acid in a second biological sample from the subject at a different time from when the first biological sample is collected; and comparing the measured amount of the homocysteic acid in the first biological sample with the measured amount of the homocysteic acid in the second biological sample.

[Item 3] A kit for use in a method according to item 1 or 2, including a reagent for measuring an amount of homocysteic acid in a biological sample from a subject.

### BRIEF DESCRIPTION OF DRAWINGS

FIG. 1a shows a scatter plot where the vertical axis indicates the homocysteic acid concentration [μM] in the plasma sample measured by competitive Enzyme-Linked Linked ImmunoSorbent Assay (ELISA). The horizontal axis indicates a negative control group of dementia (NC group: ♦), a mild cognitive impairment group (MCI group: ▴), and an Alzheimer-type dementia group (AD group: ▪).

FIG. 1b shows a scatter plot where the vertical axis indicates the homocysteic acid concentration [μM] measured by mass spectrometry. The horizontal axis indicates the negative control group of dementia (NC group: ♦), the mild cognitive impairment group (MCI group: ▴), and the Alzheimer-type dementia group (AD group: ▪).

FIG. 1c and FIG. 1d show scatter plots where their vertical axes indicate the TNF-α concentration [pg/ml] measured by ELISA and the cortisol concentration [ng/ml] measured by ELISA, respectively. The respective horizontal axis indicates the negative control group of dementia (NC group: ♦), the mild cognitive impairment group (MCI group: ▴), and the Alzheimer-type dementia group (AD group: ▪).

FIG. 2 shows a series of receiver operating characteristic (ROC) graphs to determine if the method is useful, the method for distinguishing the negative control group of dementia (NC group), the mild cognitive impairment group (MCI group), and the Alzheimer-type dementia group (AD group), respectively, from the other groups. Their horizontal axes indicate [1-specificity], and their vertical axes indicate [sensitivity].

FIGS. 2a to 2c are the ROC graphs obtained with biological samples from subjects with no medical history of brain infarction. FIGS. 2d to 2e are ROC graphs obtained with all biological samples from the subjects that include subjects with medical histories of brain infarction. FIGS. 2a and 2d are the ROC graphs to determine if the method for distinguishing between the MCI group and the NC group is useful. FIGS. 2b and 2e are the ROC graphs to determine if the method for distinguishing between the AD group and the MCI group. FIGS. 2c and 2f are ROC graphs to determine if the method for distinguishing between the NC group and a group including the MCI and AD groups.

### DESCRIPTION OF EMBODIMENTS

“Neurological disease” as used herein means any disease that causes damage to the brain, spinal cord, or peripheral nerves. Neurological diseases include, but are not limited to, brain infarction, dementia, neurodegenerative diseases such as Parkinson's disease and spinocerebellar degeneration, autoimmune neurodegenerative diseases such as myasthenia gravis and multiple sclerosis, peripheral nerve diseases such as Guillain-Barre syndrome, and muscle diseases such as muscular dystrophy.

“Dementia” as used herein means a health condition in which cognitive function that once developed normally is persistently impaired due to acquired brain damage, resulting in difficulties in daily life and social living. Dementia is generally classified into vascular dementia and degenerative dementia. The “vascular dementia” herein refers to dementia resulting from cerebrovascular disorders. Vascular dementia may be caused by, for example, brain infarction, intracerebral bleeding, subarachnoid bleeding, and hypoperfusion. Therefore, vascular dementia presents with a wide variety of imaging findings. Vascular dementia includes, but is not limited to, dementia due to lacunar infarction, dementia due to multiple cerebral microbleeds, and dementia associated with small-vessel disease. “Degenerative dementia” as used herein means cognitive dysfunction in which part or all of the brain is atrophied with cell death of nerve cells in the brain. Degenerative dementia includes, but is not limited to, Alzheimer-type dementia, Dementia with Lewy Bodies, and frontotemporal dementia. In an embodiment, the degenerative dementia is Alzheimer-type dementia. Dementia is diagnosed, for example, by a physician according to the dementia diagnostic criteria provided by NIA-AA or MMSE and/or diagnostic imaging or known biomarkers (e.g., amyloid-beta 42 in cerebrospinal fluid).

“Mild cognitive impairment (MCI)” as used herein means a health condition that is neither dementia nor cognitive normal. Mild cognitive impairment includes, but is not limited to, mild cognitive impairment due to Alzheimer's disease. Mild cognitive impairment due to Alzheimer's disease is thought to result in Alzheimer-type dementia as the conditions progress. In an embodiment, mild cognitive impairment is a mild cognitive impairment due to Alzheimer's disease. Mild cognitive impairment is diagnosed, for example, by a physician according to the dementia diagnostic criteria or the Mini-Mental State Examination (MMSE) provided by the US National Institute of Aging-Alzheimer's Association workgroup (NIA-AA).

“Brain infarction” as used herein means a health condition in which cerebral tissue becomes necrotic due to impaired blood flow in the cerebral blood vessels. Brain infarction is diagnosed, for example, by a physician based on physical findings such as abnormal eye movement, blood test results, and imaging findings. Brain infarction is, for example, classified into cardioembolic stroke and non-cardioembolic stroke.

“Cardioembolic stroke” as used herein means a brain infarction caused by the mechanism where blood clot generates in the heart, reaches the brain from the heart and occludes blood vessel in the brain. Non-cardiogenic stroke is classified into atherosclerotic brain infarction and lacunar infarction. “Atherosclerotic infarction” as used herein means a brain infarction caused by atherosclerosis in a large blood vessel in the brain. “Lacunar infarction” as used herein means a brain infarction caused by small infarctions of not more than 15 mm due to the blockage of small blood vessels in the brain.

A “subject” as used herein means a mammal. Subject may include, but is not limited to, dog, cow, sheep, non-human primate, and human. Non-human primate may include, but is not limited to, monkey, chimpanzee, orangutan, and gorilla. In an embodiment, the subject is non-human primate or human. The subject is preferably human.

A “biological sample” as used herein means a sample that may contain homocysteic acid, the sample being collected from a subject. The biological sample may be a body fluid, such as blood, lymph, urine, or the like, collected from a subject, or may be a preparation obtained by subjecting the body fluid from the subject to a predetermined treatment. The biological sample may be, for example, a blood-derived sample (hereinafter referred to as “blood sample”) or a urine-derived sample (hereinafter referred to as “urine sample”). In an embodiment, the biological sample is a blood sample.

A “blood sample” may be, for example, whole blood collected from the subject. Whole blood can be collected from a subject, for example, with a known instrument such as a needle. In one embodiment, the blood sample is a preparation such as plasma or serum. Plasma can be prepared, for example, by separating blood cell components from whole blood with a known method such as centrifugation or column chromatography. Serum can be prepared, for example, by allowing a whole blood sample containing no anticoagulant to stand at room temperature for about 20 minutes and then subjecting it to centrifugation to collect the supernatant. The blood sample may contain reagents such as anticoagulant agent.

A “urine sample” may be, for example, early morning urine, stored urine, or urine collected at any time from a subject. The urine can be collected by any known urine collection method, for example, with a cup or other container.

“Homocysteic acid (HCA)” as used herein means the amino acid shown in Formula 1 below:

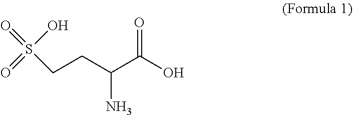 [SMILES: NC(CCS(=O)(=O)O)C(=O)O]

It is thought that accumulating homocysteic acid in the body triggers the accumulation of amyloids and leads to the development of Alzheimer-type dementia. In the blood, homocysteic acid exists in a free form or a form bound to proteins such as albumin or small molecules with thiol groups. The homocysteic acid may be a bound form of homocysteic acid that is bound to the biomolecule such as protein and lipid, or a free form homocysteic acid that is not bound to such biomolecules. In one embodiment, homocysteic acid is a bound form of homocysteic acid.

The amount of homocysteic acid in a biological sample can be measured according to known methods. The amount of homocysteic acid in the biological sample may be, for example, the weight or concentration of homocysteic acid in the biological sample or a measured value obtained by a measuring method. The concentration of homocysteic acid in a biological sample can be measured, for example, by dividing the amount of homocysteic acid in the biological sample by the volume of the measured biological sample. The molar concentration of homocysteic acid can be calculated from the weight of homocysteic acid based on its molecular weight. The amount of homocysteic acid in a biological sample can be measured, for example, by competitive ELISA.

The amount of homocysteic acid in a biological sample can be measured, for example, by ELISA. In one example, when using a competitive ELISA, homocysteic acid (HCA) in a form that can be adsorbed on the wall surface of each well of a microtiter plate, for example, HCA conjugated to bovine serum albumin (BSA) (HCA-BSA), is adsorbed in a certain amount to prepare a microtiter plate for measurement. Dispense a biological sample possibly containing homocysteic acid, a positive control solution containing a bound form of homocysteic acid at a known concentration, and a negative control solution containing no homocysteic acid to respective wells. Further, dispense an anti-homocysteic acid antibody at a predetermined amount. An antigen-antibody reaction proceeds in the mixed solution containing the reagents dispensed into each well. Remove the mixture from each well. For example, unreacted labeled antibodies and labeled antibodies reacted with the bound form of homocysteic acid at the known concentration are removed by removing the mixed solution from the wells to which the positive control solution is dispensed, while labeled antibodies bound to the homocysteic acid remain in the well. Measure the luminescence intensity of each well, which reflects the amount of labeled antibodies remaining in the wells. The difference between the light emission intensity from the well to which the negative control solution was dispensed and the light emission intensity from the well to which the positive control solution was dispensed reflects the known concentration of the added bound form of homocysteic acid. A calibration curve can be made with emission intensities from a series of known concentrations of the bound form of homocysteic acid as positive controls. The amount of homocysteic acid in the biological sample can be calculated from the light emission intensity from the well to which the biological sample is dispensed with the calibration curve.

A “mild cognitive impairment group” or “MCI group” as used herein means a group of subjects suffering from mild cognitive impairment (MCI). Subjects suffering from mild cognitive impairment in the MCI group are, for example, diagnosed by a physician according to, for example, the dementia diagnostic criteria by provided the NIA-AA or MMSE.

An “Alzheimer-type dementia group” or “AD group” as used herein means a group of subjects suffering from Alzheimer's disease (AD) type dementia. The subjects suffering from Alzheimer-type dementia in the AD group are, for example, diagnosed by a physician based on predetermined diagnostic criteria for Alzheimer-type dementia. The predetermined diagnostic criteria may be a combination of the dementia diagnostic criteria provided by the NIA-AA or MMSE and the diagnostic imaging.

A “dementia group” as used herein means a group of subjects suffering from dementia. The subjects suffering from dementia in the dementia group are diagnosed by a physician based on predetermined diagnostic criteria. The predetermined diagnostic criteria may be a combination of the dementia diagnostic criteria provided by NIA-AA or MMSE and a biomarker. In an embodiment, the dementia group is a group of subjects suffering from degenerative dementia (also referred to as the “degenerative dementia group”). The AD group described above may form a part of the degenerative dementia group. The degenerative dementia group is also referred to as a degenerative dementia group including an AD group.

A “dementia-negative control group” or “non-dementia group” (NC group) as used herein means a group of subjects who suffer or possibly suffer from a neurological disease other than dementia. In an embodiment, the NC group is a subject group that excludes a group of subjects diagnosed with dementia from a group of subjects suffering from or may have a disease causing damage to the brain, spinal cord, peripheral nerves, or the like.

Preferably, neither the “mild cognitive impairment group” nor the “dementia group” includes subjects who have or have had brain infarction. The “mild cognitive impairment group” or the “dementia group” comprises subjects who have not developed or have never developed brain infarction.

The “mild cognitive impairment group”, “dementia group”, and “dementia negative control group” may comprise, for example, no less than 20, no less than 50, no less than 100, no less than 200, or no less than 500. Each group may be further divided into subgroups based on, for example, gender, age, race, or the like. For example, the mild cognitive impairment group is further divided into subgroups such as the mild cognitive impairment group for men in their 40s and the mild cognitive impairment group for women in their 40s. For example, a subgroup is appropriately selected based on gender, age, race, or the like of the target and is used for setting a threshold value in a determination method described below.

The “sensitivity” as used herein means a quantitative index indicating whether or not a subject having a disease (positive subject) can be correctly determined to be positive in the determination method. The sensitivity is, for example, the ratio of the subjects determined to be positive in the group of positive subjects by the determination method (=[objects determined to be positive (number of persons)]/[group of positive subjects (number of persons)]).

The “specificity” as used herein means a quantitative index indicating whether or not a subject without a disease (negative control) can be correctly determined to be negative in the determination method. The specificity is, for example, the ratio of the subjects determined to be negative in the negative control group by the determination method (=[objects determined to be negative (number of persons)]/[negative control group (number of persons)]).

A threshold value for distinguishing a group from the other groups can be set, for example, by a receiver operating characteristic (ROC) graph or a ROC curve. The ROC graph or ROC curve can be set, for example, based on the balance between the sensitivity and the specificity in the determination method. The threshold value is set, for example, such that the sensitivity and specificity in the determination method are not less than a predetermined value (for example, both the sensitivity and specificity are no less than 70%, no less than 75%, or no less than 80%). In another example, the threshold value may be set to a point on a ROC graph that is close to the point (1-specificity:sensitivity) is (0:1) in the ROC graph. In another example, the threshold value may be set with the Youden index. The “Youden index” as used herein means the maximum value in the values (sensitivity+specificity−1).

In an embodiment, a threshold value for distinguishing a dementia-negative control group from a mild cognitive impairment group based on the amount of homocysteic acid in a biological sample (hereinafter, “first threshold value regarding the amount of homocysteic acid” or “first threshold value”) is lower than the threshold value for distinguishing the mild cognitive impairment group from a dementia group based on the amount of homocysteic acid in the biological sample (hereinafter “second threshold value regarding the amount of homocysteic acid” or “second threshold value”).

A ROC graph or ROC curve preferably sets the first threshold value to distinguish between a dementia-negative control group composed of subjects who have not or have never developed brain infarction and a mild cognitive impairment group composed of subjects who have not or have never developed brain infarction. Preferably, the dementia negative control group may be composed of subjects whose MMSE is 28 to 30. A ROC graph or ROC curve preferably sets the second threshold value to distinguish between the mild cognitive impairment group composed of subjects who have not or have never developed brain infarction and a dementia group composed of subjects who have not or have never developed brain infarction. In another embodiment, the first threshold value is preferably set by a ROC graph or ROC curve to distinguish between a dementia-negative control group composed of subjects who have not or have never developed brain infarction and a dementia group composed of subjects who have not or have never developed brain infarction.

“Determining” as used herein is a step that is performed semi-automatically, automatically, or mechanically without the judgment of a person with expertise such as a physician and technician.

A first aspect of the present invention provides a method for determining Alzheimer-type dementia or mild cognitive impairment, comprising: measuring an amount of homocysteic acid in a biological sample from a subject who suffers from or possibly suffers from a neurological disease and determining Alzheimer-type dementia or mild cognitive impairment based on the measured amount of the homocysteic acid. In an embodiment, the determination method comprises comparing the measured amount of homocysteic acid with the first threshold value regarding the amount of homocysteic acid and determining whether the subject has Alzheimer-type dementia or mild cognitive impairment based on the result of the first comparison.

According to the determination method in the above-described embodiment, the subject is determined to be suffering from Alzheimer-type dementia or mild cognitive impairment when the amount of the homocysteic acid is greater than the first threshold value in the first comparison where the amount of homocysteic acid in the biological sample from the subject is compared with the first threshold value.

According to the determination method in the above-described embodiment, the subject is determined to be not suffering from Alzheimer-type dementia or mild cognitive impairment when the amount of the homocysteic acid is lower than the first threshold value based on the result in the first comparison.

A determination method according to another embodiment comprises: comparing the amount of homocysteic acid with a second threshold value regarding the amount of homocysteic acid (hereinafter “second comparison”); and determining the subject to be suffered from mild cognitive impairment when the amount of the homocysteic acid is higher than the first threshold value in the first comparison, and the amount of the homocysteic acid is lower than the second threshold value in the second comparison.

In the determination method according to the above-described embodiment, the subject is determined to be suffered from Alzheimer-type dementia when the result of the second comparison is that the amount of the homocysteic acid is higher than the second threshold value.

A second aspect of the present invention provides a method for determining the progress of developing Alzheimer-type dementia or mild cognitive impairment, comprising: measuring an amount of homocysteic acid in a first biological sample collected from a subject who suffers from or possibly suffers from a neurological disease; measuring an amount of homocysteic acid in a second biological sample collected from the subject at a different time from when the first biological sample is collected; and comparing the measured amount of the homocysteic acid in the first biological sample with the measured amount of the homocysteic acid in the second biological sample. In an embodiment, the second biological sample is a biological sample of the same type as the first biological sample collected from the same subject two months after the time when the first biological sample was collected.

In an embodiment, the subject's mild cognitive impairment or Alzheimer-type dementia is determined to be progressing when the amount of homocysteic acid in the second biological sample is higher than the amount of homocysteic acid in the first biological sample.

In another embodiment, the determination method comprises measuring an amount of homocysteic acid in a third biological sample collected from the subject at a time different from the time when the second biological sample was collected, and comparing the measured amount of homocysteic acid in the first biological sample, the measured amount of homocysteic acid in the second biological sample, and the measured amount of homocysteic acid in the third biological sample. In comparing three or more measured amounts of homocysteic acid, known statistical techniques can be used to determine whether the amount tends to increase, decrease, or be stable. For example, an approximate linear function is obtained using the least-squares method from the amounts of three or more measured homocysteic acids and each measurement time. When the change (slope) of the amount of homocysteic acid concerning the measurement interval of the obtained linear function is a positive value, it can be judged that the amount of homocysteic acid tends to increase. In this case, mild cognitive impairment or Alzheimer-type dementia is determined to be progressing in the subject.

A third aspect of the present invention provides a kit used in the determination method according to the first or second aspect of the present invention. According to an embodiment, the kit includes reagents for measuring an amount of homocysteic acid in a biological sample collected from a subject. The reagents for measuring the amount of homocysteic acid include a “probe” that can specifically bind to homocysteic acid. The probe includes, for example, an antibody or a compound against homocysteic acid. The antibody is, for example, intact antibody (e.g., monoclonal antibody), antibody fragment (e.g., Fab), synthetic antibody (e.g., chimeric antibody). The antibody can be prepared by a known method, for example, immunological method, phage display method, or ribosome display method. A commercially available antibody may be used as an antibody probe. The compound against homocysteic acid includes a substance capable of specifically binding to a specific factor to be measured, for example, an aptamer. The probe may exist in free form or may be immobilized on a carrier such as beads and plates. The kit according to the second aspect may further include a buffering agent, an agent for washing, a coloring agent, and the like. The kit can be produced with the reagents according to a known method.

Reagents for measuring the amount of homocysteic acid may further include a “labeling substance” capable of generating signaling. For example, a fluorescent material and enzyme can be used as the labeling substance. The labeling substance may include, for example, known fluorescent materials and enzymes, which are commercially available or can be produced according to known methods. When an enzyme is used as the labeling substance, the reagents include a substrate corresponding to the enzyme. The substrate includes chromogenic substrate and chemiluminescent substrate. The labeling substance may be attached to the probe in advance and exist in a labeled state. Labeling may involve attaching the labeling substance to the probe directly or indirectly via at least one other substance.

A “complex” of the probe and homocysteic acid is formed by placing a plasma sample containing homocysteic acid and the reagent including a probe for homocysteic acid under a condition that allows them to contact each other. The aggregate may be separated from unreacted homocysteic acid or the detection reagent (B/F separation). When the probe includes a labeling substance, the labeling substances in the complexes generate signals reflecting the amount of homocysteic acid. The signal intensity may reflect the amount of homocysteic acid in a plasma sample when the complexes may form dependently (e.g., proportionally) as the amount of homocysteic acid increases in the plasma sample. The amount of homocysteic acid in the plasma sample can be calculated based on the signal intensity (relative value) obtained. Comparing the calculated amount of homocysteic acid with a first threshold value makes it possible to determine whether the subject, from whom the plasma sample is collected, suffers from mild cognitive impairment. The above example uses the plasma sample as a biological sample, but the biological sample according to embodiments of the second aspect is not limited to plasma sample. The biological sample may be whole blood sample, serum sample, urine sample, or other body fluids from the subject.

The particular examples will be described below. However, these examples are merely given to preferred embodiments of the present invention and do not limit the scope of the invention recited in the accompanying claims in any manner.

### EXAMPLES

### [Biological Sample]

Biological peripheral blood samples were collected from 40 subjects whom physicians diagnosed according to the Mini-Mental State Examination (MMSE). Of the 40 biological samples, 15 biological samples were collected from 15 subjects diagnosed with Alzheimer's disease (AD) and 13 biological samples were collected from 13 subjects diagnosed with mild cognitive impairment (MCI). The remaining 12 biological samples were collected from 12 subjects diagnosed with non-dementia neurological disease (NC). Each biological sample was given a sample number. The sample list recording the sample numbers includes information including the subject's age, gender, race, diagnosis result, medical history, disease complication, and MMSE score for each sample number.

### [Measurement of Homocysteic Acid Concentration by ELISA]

Each plasma sample was prepared by removing blood cell components from each biological peripheral blood sample. Each plasma sample had the corresponding sample number. The concentration of homocysteic acid (HCA) in the plasma sample was measured by a competitive immunoassay (Enzyme-Linked Immuno Substance Assay: ELISA).

A carbonate buffer (pH 9.6) containing homocysteic acid (HCA) conjugated to bovine serum albumin (BSA) (HCA-BSA) was dispensed into each well of a microtiter plate. The microtiter plate was placed at 4° C. for 48 hours or more to adsorb HCA-BSA to the respective wells. Then, the carbonate buffer was removed from the respective wells. A carbonate buffer (pH 9.6) containing BSA was dispensed into the respective wells. The microtiter plate was placed at 4° C. overnight for the blocking treatment in the respective wells, and then the carbonate buffer was removed from the respective wells. Each of the wells was washed twice with PBS-T and dried to prepare a microtiter plate for measurement.

To a micro-test tube, 50 μL of the plasma sample or a control solution containing homocysteic acid at a known concentration, 50 μL of a dilute solution, and 100 μL of a labeled-antibody solution containing an anti-homocysteic acid antibody labeled with alkaline phosphatase (ALP) (ALP-labeled anti-homocysteic acid antibody-containing solution) were dispensed and reacted at 37° C. for 120 minutes. The reaction solution was dispensed into each well of the microtiter plate for measurement and reacted at 37° C. for 120 minutes. The reaction solution was removed, and the wells were washed twice with PBS-T. The luminescent substrate, CDP-Star, was added to the respective wells of the microtiter plate after the reaction, and the luminescence intensity was measured 30 minutes later. The light emission intensities were measured from the respective samples and the control solution to calculate the HCA concentrations for the respective samples.

### [Mass Spectrometric Measurement of Homocysteic Acid Amount]

Each peripheral blood sample was treated with methanol/chloroform to precipitate protein components and remove the protein components. After the methanol/chloroform treatment, each sample was subjected to high-performance liquid chromatography (HPLC) equipped with an octadecylsilyl (ODS) column to fractionate solutions containing homocysteic acid. Components contained in the fractions were ionized by the electrospray ionization (ESI) method. The ionized components were mass-analyzed using a tandem quadrupole mass spectrometer (MS/MS).

### [Measurement of the Amount of TNF-α and Cortisol]

Tumor necrosis factor (TNF)-α is an inflammatory factor whose amount is known to increase in biological samples as the symptoms of dementia, such as Alzheimer-type dementia, progress. Cortisol is a corticosteroid whose amount is known to increase as the symptoms of dementia, such as Alzheimer-type dementia, progress. The amounts of TNF-α and cortisol in the plasma samples were measured using the Human TNF-alpha Quantikine HS ELISA (R&D Systems, inc., HSTA00E) and the cortisol ELISA (Abnova, KA0918), respectively, according to the directions in the instructions for use attached the kits. As the amounts of TNF-α and cortisol, the TNF-α and cortisol concentrations in each plasma sample were calculated with the absorbances obtained from the each plasma sample and the control solution.

### Example 1

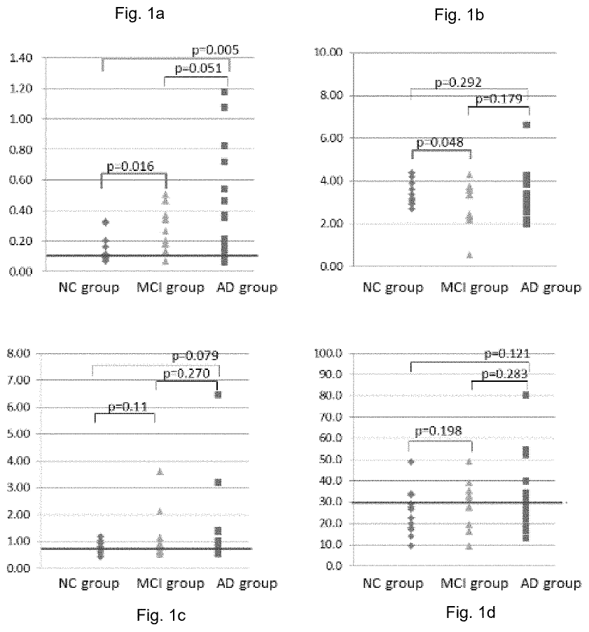

Homocysteic acid (HCA) concentration, cortisol concentration, and TNF-α concentration in a biological sample were used to examine whether a dementia negative control group (NC group), a mild cognitive impairment group (MCI group), and an Alzheimer-type dementia group (AD group) were distinguished from each other. With reference to the physicians' diagnoses in the sample list, 40 biological samples were divided into the NC group (♦), MCI group (▴), and AD group (▪), respectively. The HCA concentrations [μM] were measured by ELISA, and a scatter graph was made (FIG. 1a). The T-test was used to examine whether the NC, MCI, and AD groups were significantly distinguished based on the HCA concentration (P<0.03).

FIG. 1a shows a significant difference in the HCA concentration between the NC and MCI groups (p=0.016). This result suggests that the amount of homocysteic acid measured by ELISA can be used as an index to distinguish the NC group from the MCI group. FIG. 1a also shows a significant difference in HCA concentration between the NC and AD groups (p=0.005). This result suggests that the amount of homocysteic acid measured by ELISA can be used as an index to distinguish the NC group from the AD group. These results suggest that the amount of homocysteic acid measured by ELISA can be used as an index to distinguish the NC group from the dementia group including the MCI and AD groups.

FIG. 1a further shows a significant difference tendency in the HCA concentration between the MCI and AD groups (p=0.051). This result suggests that the amount of homocysteic acid measured by ELISA can be used as an index to possibly distinguish the MCI group from the AD group. As described below, it was possible to provide a method to determine MCI or AD with high sensitivity and specificity by excluding the biological samples collected from subjects with the disease complication or medical history of brain infarction (including lacunar infarction), which is different from dementia but can affect brain function. These results suggest that the amount of homocysteic acid measured by ELISA can be used as an index to distinguish the MCI group from the NC group or the AD group.

The HCA concentrations were measured by mass spectrometric measurement, and a scatter graph was made (FIG. 1b) in the same manner as FIG. 1a. FIG. 1b shows no significant differences in the HCA concentrations between each group. These results differ from the results shown in FIG. 1a, in which each group was significantly distinguished in the HCA concentration. Thus, different results were obtained when the HCA concentration was measured using different methods for the same plasma sample. This result is probably be caused by the difference in the form of homocysteic acid. Homocysteic acid in plasma exists in the free form in a small portion and a bound-form such as homocysteic acid bound to proteins (protein-bound from) or other thiol compounds with small-molecule weights (nonprotein-bound form) in a large portion. The ELISA (competitive method) could measure the amount of homocysteic acid in the protein-bound form, whereas the mass spectrometric measurement used in this example could not because the methanol/chloroform treatment was used to precipitate and remove the protein components. The treatment possibly affected the amount of protein-bound homocysteic acid.

The TNF-α concentrations [pg/ml] were measured by ELISA for each group, and a scatter graph was made (FIG. 1c) in the same manner as FIG. 1a. Similarly, the cortisol concentrations [ng/ml] were measured by ELISA for each group, and a scatter graph was made (FIG. 1d).

FIG. 1c shows no significant difference in the TNF-α concentration between the respective groups. FIG. 1d also shows no significant difference in the cortisol concentration between the respective groups.

### Example 2

The cognitive function may be affected by factors including a disease other than degenerative dementia. Such disease includes, for example, brain infarction. The biological samples used in Example 1 included biological samples from the subjects with a disease complication or medical history of brain infarction. In Example 2, 30 biological samples were selected by checking the sample list and excluding biological samples from the subjects with disease complications or medical histories of brain infarction. The 30 biological samples were used to examine whether the MCI group, the AD group, or a group including the MCI and AD groups and the NC group were distinguished based on homocysteic acid (HCA) concentrations measured by ELISA.

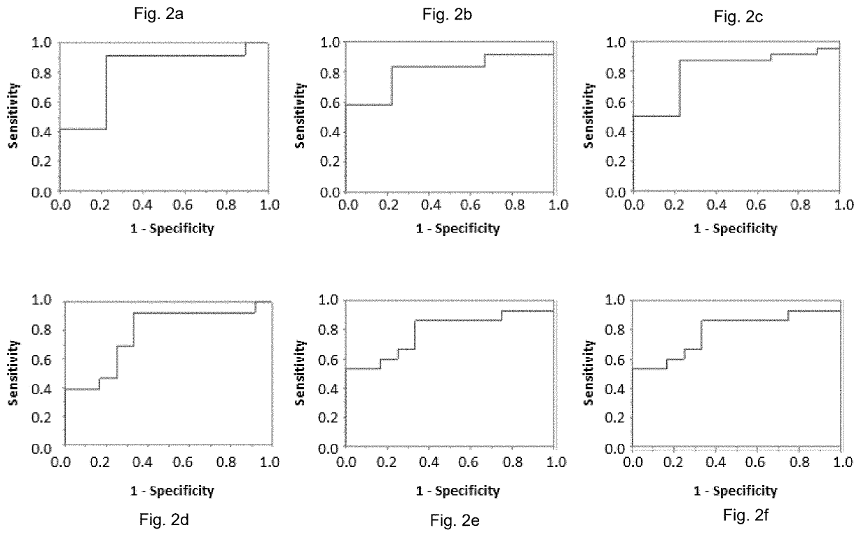

Using a receiver operating characteristic (ROC) curve, a determination method was examined to effectively distinguish the MCI group from the NC group (FIG. 2a). The ROC area was 0.815 in FIG. 2a. A point on the ROC curve having a short distance from the point where (1-specificity:sensitivity) is (0:1) was used as a threshold value (=0.116) for the determination method. At this point, the sensitivity was 92%, and the specificity was 78%.

Similarly, the effectiveness of a method for distinguishing the AD group from the NC group was examined with a ROC curve (FIG. 2b). The result indicates that the sensitivity was 83%, and the specificity was 78%. Further, the effectiveness of a method for distinguishing the group including the MCI and AD groups from the NC group was examined with a ROC curve (FIG. 2c). The result indicates that the sensitivity was 88%, and the specificity was 78%.

Table 1 indicates the results in Example 2.

TABLE 1

| | Items | | | | | |
|---|---|---|---|---|---|---|
| | Example 2 | | | Comparative example | | |
| | Diseases to be distinguished | | | | | |
| | MCI | AD | MCI + AD | MCI | AD | MCI + AD |
| threshold value | 0.116 | 0.116 | 0.116 | 0.116 | 0.116 | 0.116 |
| amount | 92% | 83% | 88% | 92% | 87% | 89% |
| sensitivity | 78% | 78% | 78% | 67% | 67% | 67% |
| ROC area | 0.815 | 0.806 | 0.810 | 0.782 | 0.789 | 0.786 |
| FIG. 2 | a | b | c | d | e | f |

As comparative examples, all the 40 biological samples, including the biological samples from the subjects with the disease complication or medical history of brain infarction, were used to examine the effectiveness of the respective determination methods in the same manner as Example 2. The sensitivity of the method for distinguishing the AD group from the NC group was 92%, and the specificity was less than 70%, examined with the use of a ROC curve (FIG. 2d). Similarly, using a ROC curve, the determination methods were examined to effectively distinguish the AD group from the NC group and distinguish the group including the MCI and AD groups and the NC group (FIGS. 2e and 2f). The sensitivities were 87% and 89%, respectively, and both specificities were below 70% (Table 1).

Example 2 demonstrates that the determination method, in which the amount of homocysteic acid in a biological sample such as blood collected from a subject who has not or has never developed brain infarction is used as an indicator, can distinguish an MCI group, an AD group, or a group including MCI and AD groups from the NC group with the sensitivity and specificity of more than 70%.

1. A method for determining Alzheimer-type dementia or mild cognitive impairment, comprising:
measuring an amount of homocysteic acid in a biological sample from a subject who suffers from or possibly suffers from a neurological disease, and
determining the subject to suffer from Alzheimer-type dementia or mild cognitive impairment based on the measured amount of the homocysteic acid.

2. The method according to claim 1, wherein in the determining, the subject is determined to suffer from Alzheimer-type dementia based on the measured amount of the homocysteic acid.

3. The method according to claim 1, wherein in the determining, the subject is determined to suffer from mild cognitive impairment based on the measured amount of the homocysteic acid.

4. A method for determining the progress of developing Alzheimer-type dementia or mild cognitive impairment, comprising:
measuring an amount of homocysteic acid in a first biological sample from a subject who suffers from or possibly suffers from a neurological disease;
measuring an amount of homocysteic acid in a second biological sample from the subject at a different time from when the first biological sample is collected; and
comparing the measured amount of the homocysteic acid in the first biological sample with the measured amount of the homocysteic acid in the second biological sample.

5. The method according to claim 1, wherein the homocysteic acid is a bound form of homocysteic acid in the biological sample.

6. The method according to claim 1, wherein the biological sample is a blood or urine sample.

7. The method according to claim 1, wherein the subject is a subject who has not suffered or has never suffered from brain infarction.

8. A kit for use in a method according to claim 1, including a reagent for measuring an amount of homocysteic acid in a biological sample from a subject.

9. The kit according to claim 8, wherein the homocysteic acid is a protein-bound form of homocysteic acid in the biological sample.

10. The kit according to claim 8, wherein the subject is a subject who has not suffered or has never suffered from brain infarction.

11. The method according to claim 4, wherein the homocysteic acid is a bound from of homocysteic acid in the biological sample.

12. The method according to claim 4, wherein the biological sample is a blood or urine sample.

13. The method according to claim 5, wherein the biological sample is a blood or urine sample.

14. The method according to claim 11, wherein the biological sample is a blood or urine sample.

15. The method according to claim 4, wherein the subject is a subject who has not suffered or has never suffered from brain infarction.

16. A kit for use in a method according to claim 4, including a reagent for measuring an amount of homocysteic acid in a biological sample from a subject.
